(19) United States
(12) Patent Application Publication
(10) Pub. No.: US 2011/0160508 A1
(43) Pub. Date: Jun. 30, 2011

(54) PRODUCTION OF AROMATICS FROM METHANE

(76) Inventors: Ding Ma, Liaoning (CN); Lijun Gu, Liaoning (CN); Xinhe Bao, Liaoning (CN); Wenjie Shen, Liaoning (CN); Martin Philip Atkins, Middlesex (GB)

(21) Appl. No.: 12/736,899

(22) PCT Filed: May 21, 2008
(86) PCT No.: PCT/CN2008/000978

§ 371 Date: Mar. 1, 2011

(51) Int. Cl. C07C 2/76; B01J 29/06; B01J 29/40; B01J 29/076

(52) U.S. Cl. 585/415; 502/64; 502/71

(57) ABSTRACT

A catalytic composition and method for methane dehydroaromatisation, the catalytic composition comprising a catalyst metal active for methane dehydroaromatisation, a zeolite having pores with diameters of at least 10 non-oxygen frame-work atoms, and silicon carbide, and in which the method comprises contacting a methane-containing feedstock with said catalytic composition to produce one or more aromatic compounds and hydrogen.

This invention relates to the production of aromatic compounds from methane, more specifically to a process for producing aromatic compounds and hydrogen from methane in the presence of catalysts comprising aluminosilicate zeolites.

Methane, typically derived from natural gas, is an attractive fuel for power generation due to its very clean burning properties, and its high energy content per unit mass. However, a problem with methane is that it is relatively complex to transport compared to liquid or solid fuels such as crude oil or coal. Pipeline infrastructure is generally required for providing a continuous supply of natural gas to a regional supply network. The alternative is to transport it in the form of liquefied natural gas, typically by ship or by rail. Gas liquefaction requires energy intensive compression and cryogenic equipment. Additionally, re-gasification facilities at the unloading terminals are required.

The use of methane for generating chemicals or liquid fuels is becoming increasingly attractive, as manufacturing sites can be based at a natural gas source, and the methane-derived liquid products can be readily transported to their point of use by conventional liquid, transportation means, without the need for, compression and cryogenic liquefaction facilities.

Large volume products of methane include Fischer Tropsch-derived hydrocarbon fuels and methanol, both of which are produced by first converting the natural gas into syngas (a mixture of carbon monoxide and hydrogen) and then converting the syngas into the desired products.

However, to avoid the necessity to use two separate manufacturing steps, single step processes for converting methane to fuels and chemicals products are being increasingly studied.

One known process is the dehydroaromatisation of methane to produce aromatic hydrocarbons, henceforth termed “aromatics”, which are widely used to produce chemical precursors and products. One major use of aromatics is in the production of purified terephthalic acid (PTA), which is a feedstock for the production of, inter alia, polyester plastics and fibres.

U.S. Pat. No. 4,239,658 describes how a mixture of ethylene and benzene is produced when methane is contacted with a catalyst comprising a Group IB or Group VIII metal, a Group VIB metal oxide, and a Group IIA metal. Additionally, Xu et al in J. Catal., 216 (2003); 386-95 report the use of Mo-containing ZSM-5 catalysts (Mo/ZSM-5) for producing aromatic compounds from methane.

However, a problem with methane dehydroaromatisation, particularly under non-oxidative conditions, is that the catalytic activity can degrade with time. This is thought to occur through the formation of coke or other carbonaceous deposits on the catalyst, which are thought to arise from metal-carbene intermediates that have been postulated as being formed during the catalytic cycle.

Iglesia et al in J. Catal., 206 (2002), 14-22 report that deactivation of a Mo/ZSM-5 catalyst in non-oxidative methane dehydroaromatisation can be reduced by silylation to remove Bronsted acid sites on the external crystal surfaces of the zeolite.

Lin et al, in J. Phys. Chem., B; 2002, 106, pp 8524-30 report that catalyst coking and deactivation in Mo/ZSM-5 catalysts can also be reduced by partially dealuminating the ZSM-5 zeolite.

However, there remains a need for an alternative process for producing aromatic compounds from methane. Additionally, there remains a need for a catalyst for producing aromatic compounds from methane having improved selectivity to aromatic compounds, and with improved resistance to deactivation.

According to a first aspect of the present invention, there is provided a catalyst composition active for methane dehydroaromatisation reactions, which composition comprises a catalyst metal active for methane dehydroaromatisation and a zeolite with Brønsted acidity, which zeolite is selected from those having pores in one or more dimensions with diameters of at least 10 non-oxygen framework atoms, characterised by the composition also comprising silicon carbide.

According to a second aspect of the present invention, there is provided a process for methane dehydroaromatisation comprising contacting a methane-containing feedstock with a catalyst to produce one or more aromatic compounds and hydrogen, which catalyst comprises a catalyst metal active for methane dehydroaromatisation, and a zeolite with Bronsted acidity, which zeolite is selected from those having pores in one or more dimensions with diameters of at least 10 non-oxygen framework atoms, characterised by the catalyst also comprising silicon carbide.

Methane dehydroaromatisation requires the presence of one or more catalyst metals which are active for removing hydrogen from methane. Although not wishing to be bound by theory, it is believed that the activity of the one or more catalyst metals results from their ability to form metal carbide species (M=C) under the reaction conditions. Typically, the catalyst metal is selected from one or more of Mo, W and Re. Mo-containing catalysts are preferred. The catalyst metal content of the catalyst is typically present in the zeolite at a loading in the range of from 0.1 to 20 wt %, for example in the range of from 1 to 10 wt %.

Without being bound by any theory, it is thought that benzene formation by dehydroaromatisation of methane occurs via a two-step mechanism. Firstly, methane is dehydrogenated over the catalyst metal of the catalyst to form hydrogen and metal-carbide species, M=C. The M=C species then act as centres for reaction with further methane molecules to produce ethylene, C₂H₄. In the presence of Brønsted acid sites, condensation of ethylene molecules to higher hydrocarbons, in particular benzene and other aromatic compounds, is catalysed.

Optionally, the catalyst can comprise one or more additional metals. Where Mo is the catalyst metal, preferred additional metals include one or more of Ru, Pt, W, Ze, Co, Fe and Cr. Where W is the catalyst metal, Zn is a preferred additional metal.

Where present, additional metals are typically present in the zeolite at a loading in the range of from 0.1 to 20 wt %, for example in the range of from 0.1 to 10 wt %.

Zeolites that can be used in the present invention include those having pores with diameters formed of at least 10 non-oxygen framework atoms in one or more dimensions. For brevity, such a pore diameter will henceforth be referred to as a 10-membered ring. Pore structures with diameters smaller than 10-membered rings are believed to be too small to allow passage of aromatic compounds. A database of zeolite structures is maintained by the International Zeolite Association.

Examples of suitable zeolite structures include MFI and MWW structures, which both have 10-membered ring pore diameters. In the case of MFI, the pore structure is three dimensional. There are two channel systems having 10-membered ring diameters. One of the 10-membered ring channels or pores is linear, the other is sinusoidal. In the case of the MWW structure, the pore structure is two-dimensional, the pores in each dimension being formed of 10-membered rings. The pores intersect at cages which are formed from 12-membered rings.

The zeolites of the process of the present invention have Brønsted acid characteristics, in which the charge on the zeolite framework is negative. The Brønsted acidity arises where protons or H₃O⁺ ions act as the counter-cations to the framework negative charge. Such framework negative charge is found in aluminosilicate and silicoaluminophosphate zeolites, for example. The zeolite framework can additionally comprise other elements, such as boron, cobalt, titanium, gallium or germanium. Aluminosilicate zeolites tend to exhibit stronger acidity compared to silicoaluminophosphates, for example. This is advantageous for methane dehydroaromatisation, as methane conversions are typically higher in the presence of stronger acids. In a preferred embodiment of the invention, the zeolite is an aluminosilicate zeolite adopting the MWW of MFI structure, for example MCM-22, MCM-49 or ZSM-5. The silicon/aluminium molar ratio of aluminosilicate zeolites is suitably in the range of from 1 to 150, and is preferably in the range of from 15 to 40.

Catalysts comprising SiC have been shown to have higher methane conversions per mole of catalyst metal compared to the SiC-free catalysts. In the case of the ZSM-5-containing catalysts, it has been found that improved catalyst lifetime results when the catalyst comprises silicon carbide, compared to corresponding catalysts which do not comprise silicon carbide.

The one or more catalyst metals can be incorporated into the zeolite during synthesis of the zeolite, or by modifying the zeolite after its synthesis, typically through ion exchange or by impregnation. It has been found that the most active form of the catalyst metal is where it is present as counter-cation to the negative framework charge. In condensed form, for example where the catalyst metal is in the form of metal-oxide particles located either within the internal zeolite structure or on the external surface of the zeolite structure, the catalyst metal shows low activity. Therefore, in a preferred embodiment of the invention, the catalyst metal is present, at least in part, in the form of discrete ions within the zeolite channel or pore structure, which exhibit higher catalytic activity compared to small catalyst metal oxide particles.

The zeolite content of the catalyst is typically in the range of from 0.5 to 40% by weight.

A typical zeolite synthesis mixture comprises sources of the framework atoms, and a so-called structure-directing agent, usually an organo-amine compound. The structure directing compound is provided in one embodiment in the form of a hydroxide, for example a quarternary ammonium hydroxide having one to four organic groups on the nitrogen atom. Additionally, or alternatively, other hydroxides can be present, for example inorganic hydroxides such as sodium or potassium hydroxide, or other quarternary ammonium hydroxides having one to four organic groups on the nitrogen atom.

The source of framework atoms can be in the form of small oxide particles, for example in the form of a colloidal suspension, or in the form of one or more water soluble compounds or salts, for example alkoxide compounds, halide salts, oxalate salts, carbonate salts or nitrate salts.

The one or more catalyst metals and/or optional additional metals can also be present in the zeolite synthesis mixture, being present in the form of one or more soluble compounds or salts, such as alkoxide compounds, as halide salts, as oxalate salts, as carbonate salts or as nitrate salts.

Alternatively, the one or more catalyst metals and/or optional additional metals can be incorporated into the catalyst after the calcination stage, for example through impregnation or ion exchange techniques. Such procedures can be carried out either on the zeolite itself, or on a zeolite/silicon carbide composite.

Ion-exchange can be achieved by suspending the zeolite in a solution of the one or more catalyst metals, optionally at elevated temperature and/or pressure, followed by filtration and drying. This procedure can then be repeated if necessary until the desired loading of catalyst metal(s) is achieved.

Impregnation can be carried out by suspending the zeolite in a solution of the one or more catalyst metal(s), and then evaporating the solution to dryness.

In zeolite synthesis, the synthesis mixture is typically an aqueous mixture comprising sources of the zeolite framework constituent elements. For aluminosilicate zeolites, the synthesis mixture comprises sources of silicon and aluminium. These are either dissolved in the (usually aqueous) solvent or are suspended therein. Silicon is often provided in the form of a tetraalkoxysilane such as tetraethoxysilane, or as a sodium silicate solution or silica or silicate colloid. Examples of suitable aluminium sources include aluminium chloride and sodium aluminate.

The zeolite synthesis mixture typically additionally comprises one or more organoamine salts, often hydroxides, which can act as structure-directing agents for the zeolite. Additionally, ammonia or an organoamine hydroxide salt is often added to adjust pH. The pH of zeolite synthesis solutions is typically in the range of from 8 to 11, for example from 9 to 10. Additional amines or organoamine salts can also be added as structure directing agents. In ZSM-5 synthesis, for example, one or more tetrapropylammonium salts, typically hydroxide, are added to the synthesis mixture.

The catalyst comprises silicon carbide (SiC). The SiC can be in the form of particles, or can be in the form of a monolith or foam. Silicon carbide has a high thermal, mechanical and chemical stability and a low expansion coefficient, which makes it resistant to degradation and also thermal stresses that can be experienced under the high temperature conditions of methane dehydroaromatisation.

In one embodiment of the invention, the SiC is foam or sponge-like, comprising bubble-like pores or cavities within the structure that impart increased surface area to the material. This is advantageous, as increased zeolite loadings in the zeolite/SiC composite materials can be achieved. Such foam SiC materials can be prepared by the method described in U.S. Pat. No. 4,914,070, for example, in which SiO vapours formed from a mixture of silica and silicon at 1100-1400° C. in one reaction zone are passed over carbon in a separate reaction zone at 1100-1400° C. It has been found that foam materials can also be achieved by mixing together SiC powder and carbon powder, e.g. graphite, and calcining the mixture in an oxygen-containing atmosphere at a temperature in excess of 950° C., and preferably at a temperature of 1000° C. or more, for example 1100° C. or more, such as 1400° C. or more. The temperature is also suitably maintained at 1600° C. or less, for example 1500° C. or less. Suitable temperature ranges for the calcination are in the range of from 1100 to 1600° C., for example in the range of from 1400 to 1500° C. Optionally, the powders can be first suspended or mixed to a paste in a liquid, such as water or ethanol, and allowing the liquid to evaporate off to dryness, optionally under conditions of elevated temperature, before calcination. The oxygen-containing atmosphere can be pure oxygen or air. This method requires only a single reaction zone, and hence requires less complex equipment compared to the two reactor zone requirements of U.S. Pat. No. 4,914,070, for example. This method of producing foam-like SiC is described in a co-pending patent application.

Typically, the particle sizes of the silicon carbide and carbon materials are chosen so that the carbon particles are larger than the silicon carbide particles. In one embodiment, the average diameter of the carbon particles is at least ten times that of the silicon carbide particles, and in a further embodiment at least 50 times that of the silicon carbide particles.

Typically, the average diameter of the silicon carbide particles is up to 50 μm and at least 0.05 μm. In one embodiment, the average diameter of the silicon carbide particles is μm or less, such as 1 micron or less. In a further embodiment, the silicon carbide particles have an average particle diameter of 0.5 μm.

The carbon particles typically have an average diameter of up to 100 μm, and at least 0.1 μm. In one embodiment, the average particle diameter of the carbon is greater than 10 μm, for example greater than 20 μm. In a further embodiment the carbon particles have an average particle diameter of 32 μm.

The weight ratio of silicon carbide to carbon particles is typically in the range of from 10:1 to 1:10, for example in the range of from 4:3 to 1:10, such as in the range of from 1:1 to 1:5. Lower silicon carbide to carbon weight ratios tend to favour a more porous, open resulting silicon carbide structure with increased pore volume.

The silicon carbide can be incorporated into the catalyst by a variety of techniques. In one embodiment, this is achieved by mechanically mixing particulate silicon carbide with the zeolite or metal-modified zeolite. In another embodiment, SiC is added to a zeolite synthesis mixture, such that zeolite crystals deposit on the SiC particles. To increase zeolite loadings, the zeolite-coated SiC particles can be re-suspended in zeolite synthesis mixture one or more additional times until the desired zeolite content is reached. In a further embodiment, a dip-coating method is used, in which silicon carbide is added to a so-called “polycation” solution, such as polymethylacrylamide or poly(diallyldimethylammonium chloride), polyethylenimine or polyacrylic acid, before being dried and subsequently suspended in a solution of zeolite synthesis mixture. This process is optionally repeated one or more times, and the resulting solid is calcined in air to produce a zeolite-coated silicon carbide composite.

In methane dehydroaromatisation, a feedstock comprising methane is contacted with the catalyst under conditions of elevated temperature and optionally elevated pressure. The reaction temperature is suitably in the range of from 400 to 900° C., and is typically in the range of from 600 to 850° C. The pressure is typically in the range of from 1 to 80 atm, for example in the range of from 1 to 50 atm, such as in the range of from 1 to 25 atm. Optionally, methane is not the sole component of the feedstock, and in one embodiment an inert diluent such as nitrogen or argon is additionally present. In a further embodiment, where a diluent is present, the methane concentration in the feedstock is in the range of from 0.1 to 20% by volume.

The catalyst is typically in the form of a fixed bed, with the methane-containing feedstock being passed over the catalyst. Typically, the GHSV (Gas Hourly Space Velocity, in units of mL gaseous feedstock corrected to standard temperature and pressure, per g catalyst, per hour) of the total feedstock is in the range of from 100 to 20 000 mL g⁻¹ h⁻¹, for example in the range of from 100 to 10 000 mL g⁻¹ h⁻¹, and more preferably in the range of from 1 000 to 5 000 mL g⁻¹ h⁻¹, such as 1000 to 2000 mL g⁻¹ h⁻¹.

The products of the reaction are one or more aromatic compounds and hydrogen. The aromatic compounds that can be produced in the reaction include benzene, toluene, one or more xylene isomers (often referred to collectively as “BTX”). By-products include double-ring aromatic compounds such as naphthalene and aliphatic hydrocarbons. Additionally, carbonaceous deposits can also be produced which can cause or contribute to catalyst fouling or coking.

In a preferred embodiment of the present invention, the zeolite is ZSM-5. Catalysts comprising ZSM-5 show higher catalytic activity compared to MCM-22 containing catalysts, for example. In addition, catalysts made from ZSM-5 show enhanced resistance to deactivation when the catalyst comprises silicon carbide.

A typical synthesis of ZSM-5 is to prepare an aqueous ZSM-5 synthesis mixture comprising a source of silicon, a source of aluminium, and tetrapropylammonium hydroxide, and heating the mixture in a sealed vessel to a temperature in the range of from 100 to 300° C., typically in the range of from 150 to 250° C. As a result of the heating, the pressure in the sealed vessel increases to above ambient, the final pressure being dependent on the composition of the zeolite synthesis mixture, on the temperature, and on the air space in the sealed vessel that is not initially occupied by the zeolite synthesis mixture. Typically, the zeolite crystallises over a period of several hours, for example in the range of from 1 to 200 hours. The catalyst is then separated from the zeolite synthesis mixture, typically by filtration, decantation or centrifugation.

The resulting solid is then typically washed with water to remove excess zeolite synthesis mixture. Often, washing is continued until the pH of the wash-water falls below a certain level, for example below pH 8 or until pH 7 is reached. The washed solid can then be dried, typically at above-ambient temperatures such as up to 200° C., to remove residual water. The zeolite can then be calcined in an oxygen-containing atmosphere at temperatures usually in the range of from 450 to 650° C. to burn-off any organic material, for example organoamine components originating from the zeolite synthesis mixture.

A zeolite/silicon carbide composite can, in one embodiment, be prepared by adding silicon carbide particles to the zeolite synthesis mixture before the hydrothermal synthesis, which results in zeolite crystals depositing on the surface of the silicon carbide when subjected to hydrothermal synthesis conditions.

In an alternative embodiment, particles of zeolite and silicon carbide can be mixed mechanically to form the composite, for example by suspending zeolite and silicon carbide particles in water and allowing the suspension to evaporate to dryness.

The one or more catalyst metals and/or additional metals can either be added to the zeolite synthesis mixture in the appropriate quantity, typically in the form of a water-soluble compound for example as a nitrate, carbonate or oxylate salt. The catalyst metal(s) and optional additional metals can then become incorporated into the zeolite during hydrothermal synthesis.

The zeolite-containing catalyst can also be modified to increase acidity. Often zeolites after synthesis contain alkali-metal ions as the counter-ions to the framework negative charge. These can be replaced with protons, for example by washing with acid, or by washing with an ammonium salt and calcining the product. This acts to provide some control over the number and concentration of Brønsted acid sites in the zeolite.

There now follow non-limiting examples illustrating the invention, with reference to the Figures in which;

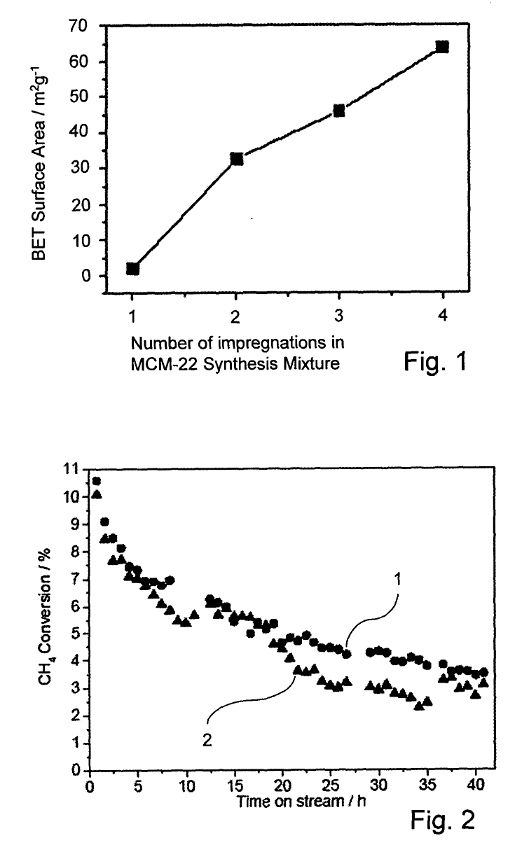

FIG. 1 is a graph showing the BET surface area of MCM-22/SiC composites of materials made after repeated immersion of SiC and MCM-22/SiC composites in an MCM-22 synthesis mixture.

FIG. 2 is a graph showing CH₄ conversions in the presence of Mo/MCM-22 and Mo/MCM-22/SiC catalysts.

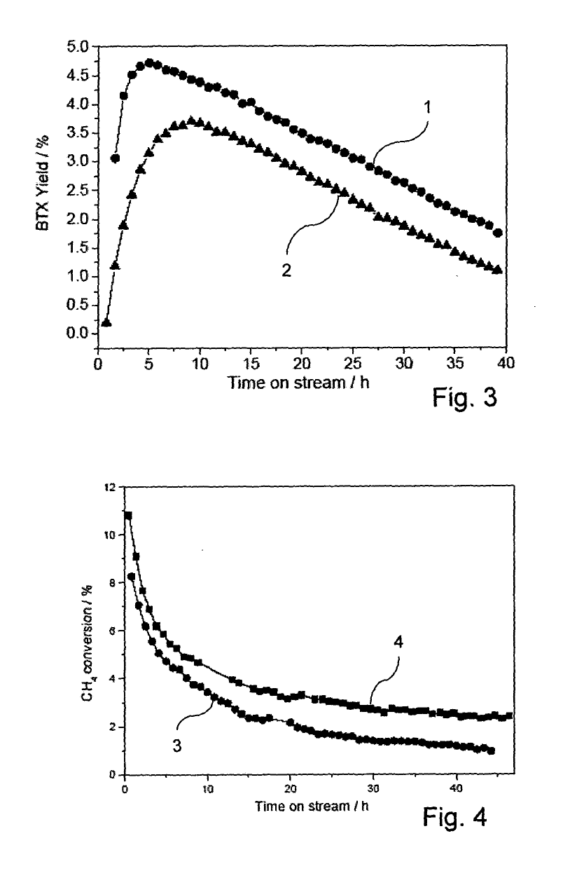

FIG. 3 is a graph showing BTX yields in the presence of a Mo/MCM-22 catalyst and a Mo/MCM-22/SiC catalyst.

FIG. 4 is a graph showing CH₄ conversions in the presence of Mo/ZSM-5 and Mo/ZSM-5/SiC catalysts.

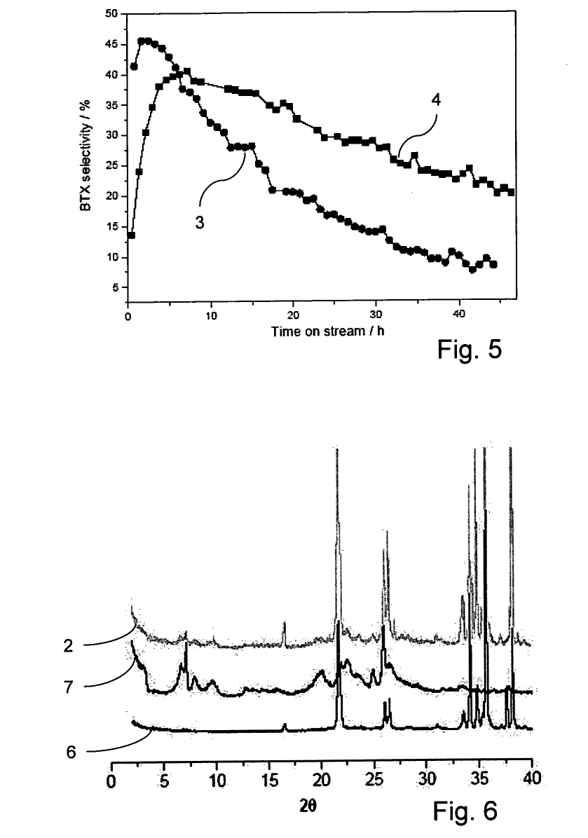

FIG. 5 is a graph showing BTX selectivity in the presence of a Mo/ZSM-5 catalyst and a Mo/ZSM-5/SiC catalyst.

FIG. 6 shows XRD patterns of MCM-22, SiC and Mo/MCM-22/SiC catalysts.

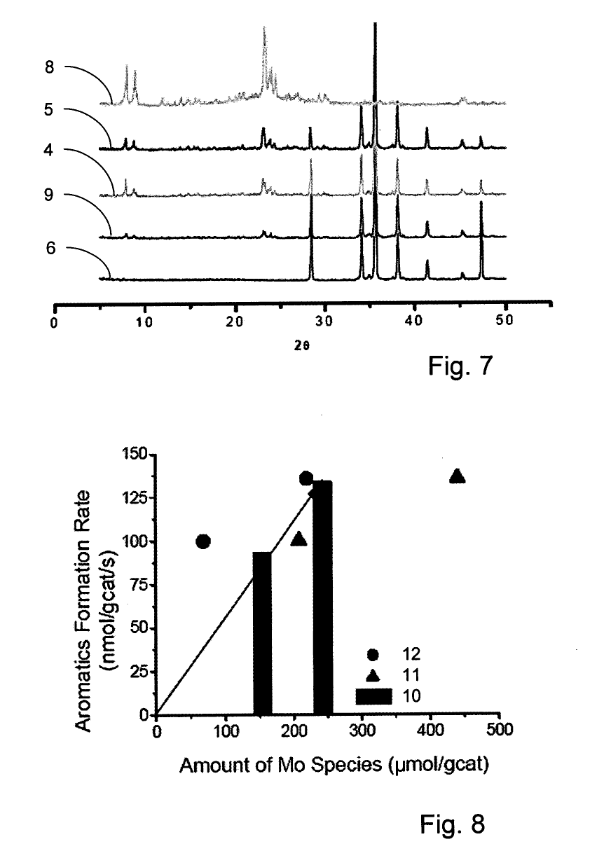

FIG. 7 shows XRD patterns of ZSM-5, SiC and Mo/ZSM-5/SiC catalysts before, during and after use.

FIG. 8 is a graph which shows catalyst activity as a function of the different types of molybdenum species present in the catalyst.

Non-porous silicon carbide powder from Shandong Qingzhou Micropowder Co. Ltd was used in the following experiments.

In the following examples, the silicon carbide was pre-treated in air at 900° C. for 2 hours before use.

### EXAMPLE 1

### Mo/MCM-22/SiC

An MCM-22 zeolite synthesis mixture was prepared by mixing the components listed below in the stated molar ratios:

Na₂O: 13.5

Al₂O₃: 3.3

SiO₂: 100

H₂O: 4500

Hexamethyleneimine (HMI): 50

The Si/Al molar ratio of the synthesis mixture was 15.

The mixture was stirred at 30° C. for 3 hours, and 60 g of the solution together with 8 g pre-treated silicon carbide (10-20 mesh particle size) were transferred to a polytetrafluoroethylene (PTFE)-lined autoclave, sealed and heated to a temperature of 150° C., and maintained at that temperature for 168 hours before being allowed to cool. The product was filtered off, and added at least once to another 60 g of the zeolite synthesis mixture in an autoclave, which was sealed and heated again to 150° C., and maintained at that temperature for a further period of 168 hours. Increased MCM-22 loadings were achieved by repeating further this step of separating the MCM-22/SiC composite and resuspending it in further MCM-22 synthesis gel.

After cooling, the solid product was filtered off, washed with deionised water and dried at 120° C. The solid was then calcined in air at a temperature of 540° C. for 10 hours. To produce the acid form of the zeolite, 1 g of MCM-22/SiC composite underwent two successive ion-exchanged treatments with 150 to 180 mL 0.4M NH₄NO₃ for 3 hours at a temperature of 80° C., with the solid being filtered off between the two treatments. The resulting ammonium-exchanged MCM-22/SiC composite was then again calcined in air at 540° C. Loadings of MCM-22 on SiC achieved by this method were typically in the range of from 4 to 20 wt %).

Control over the loading of MCM-22 on SiC could be achieved by varying the number of times the SiC or MCM-22/SiC composite was suspended in an MCM-22 synthesis mixture. This is shown in Table 1, which highlights the increase in loading of MCM-22 on SiC with repeated suspension of SiC and MCM-22/SiC composites using the above-described MCM-22 synthesis mixture and synthesis conditions. Further control of the MCM-22 loading can be achieved by varying the length of time that the SiC or SiC/MCM-22 composite was suspended in the synthesis mixture.

TABLE 1
MCM-22 content of MCM-22/SiC composite with repeated suspension
in MCM-22 synthesis mixture.

| Number of repeated suspensions in | Weight Increase |
|---|---|
| MCM-22 synthesis mixture | of SiC (%)^{a} |
| 1 | 0 |
| 2 | 4-8.5 |
| 3 | 11.5-16.6 |
| 4 | 18.5 |

^{a}compared to the initial mass of SiC used.

As shown in FIG. 1, the number of times the SiC or MCM-22/SiC composite is immersed in MCM-22 synthesis mixture also has an effect on the BET surface area of the calcined materials, the surface area typically increasing from a value of about 1 to 2 m²g⁻¹ after one immersion to a value of from 60 to 70 m² g⁻¹ after four immersions.

Molybdenum was incorporated into the zeolite by adding an aqueous solution of ammonium heptamolybdate (NH₄)₆[Mo₇O₂₄].4H₂O to the MCM-22/SiC composite by incipient wetness. The volume and concentration of the molybdate solution was calculated so as to provide a Mo loading of 6 wt %, based on the zeolite content. This was calculated by adding water in a sufficient volume to fill the void space of the materials, and to just cover the solid with solution, and adding the appropriate mass of molybdate that corresponded to a Mo loading on the zeolite portion of the solid of 6 wt %. The suspension was allowed to dry at room temperature for 12 hours, followed by a further 2 hours at 120° C. to ensure evaporation of all water, to leave a molybdage-impregnated solid. The resulting material was then calcined in air at 500° C. for 6 hours.

### COMPARATIVE EXAMPLE 2

MCM-22 was prepared in an analogous way to that of Example 1, except that no silicon carbide was added to the MCM-22 synthesis mixture. The Si/Al molar ratio in the synthesis mixture was 15.

### EXAMPLE 3

A ZSM-5 synthesis mixture was prepared by mixing the components listed below in the stated molar ratios:

Na₂O: 0.1

SiO₂: 3.6

Al₂O₃: 0.072

H₂O: 60

Tetrapropylammonium hydroxide (TPAOH): 0.66

The Si/Al molar ratio of the synthesis mixture was 25.7.

The mixture was stirred for 3 hours at 30° C. 20 g of the ZSM-5 synthesis mixture and 8 g pre-treated SiC (10-20 mesh particle size) were transferred into a PTFE-lined autoclave, which was sealed and heated to a temperature of 180° C. This temperature was maintained for 48 hours before being allowed to cool. The resulting solid was washed with deionised water, dried at 120° C., and calcined in air at 550° C. for 5 hours.

The ZSM-5/SiC composite was twice ion-exchanged with 0.4M NH₄NO₃, in each case for a period of 3 hours at 80° C. The ammonium-exchanged ZSM-5/SiC composite was then calcined in air at 540° C. for 5 hours, to convert the ZSM-5 component of the composite to the protonated form. The quantity of zeolite in the catalyst was 6.2 wt %.

Molybdenum was added to the catalyst in the same way as Example 1, the quantity of molybdenum in the aqueous ammonium molybdate solution being sufficient to give a molybdenum loading of 6% by weight.

### COMPARATIVE EXAMPLE 4

An Mo/ZSM-5 catalyst was prepared in an analogous way to the composite catalyst of Example 3, except that no silicon carbide was added to the initial ZSM-5 synthesis mixture. Mo impregnation was carried out so as to give a Mo loading of 6 wt % of the zeolite.

### EXAMPLE 5

5 g of the Mo/ZSM-5 catalyst of Comparative Example 4 was suspended in 6 mL deionised water and stirred. 3.836 g of pre-treated SiC (20-40 mesh) was added under stirring. The suspension was then left to dry at 120° C. to produce a Mo/ZSM-5/SiC composite. The quantity of zeolite in the catalyst was 5.6 wt %.

Catalyst structure was analysed using X-Ray Diffraction (XRD), which confirmed the presence of MCM-22, ZSM-5 and SiC phases in the respective compositions.

Catalytic experiments were conducted using a fixed-bed reactor, down-flow quartz tube reactor, having an inner diameter of 8 mm. Reactions were conducted under atmospheric pressure. The mass of catalyst used was such that the mass of the Mo/Zeolite component was 0.1 g. The catalyst particle size was 20-40 mesh, i.e. between 0.85 and 0.42 mm. The catalyst was pre-heated in helium while the temperature was ramped to 700° C., and a feed gas comprising methane and nitrogen (CH₄:N₂ molar ratio of 9:1) at a rate of 25 mL min⁻¹ was passed over the catalyst.

The products were analysed by on-line GC, using a Varian CP-3800 device equipped with both FID and TCD detectors. The nitrogen was used as the internal standard to quantify the methane converted and the yield of products.

For Mo/MCM-22-containing catalysts, yields of BTX were typically between 6 to 8 times that of two-ring aromatic compounds such as naphthalene on a molar basis. For Mo/ZSM-5-containing catalysts, the BTX yield was about 10 times higher than two-ring aromatic compounds.

As can be seen from FIG. 2, there is little or no negative effect on methane conversion when equivalent masses of Mo/MCM-22, 1, and Mo/MCM-22/SiC, 2, catalysts are employed in methane dehydroaromatisation. Although from FIG. 3 the BTX yields are not as high as the SiC-free Mo/MCM-22 catalyst, it also shows that there is no increased rate of deactivation of the catalyst.

FIG. 4 demonstrates that, in the case of ZSM-5-containing catalysts, methane conversion is higher for the Mo/ZSM-5/SiC catalyst, 4, compared to the SiC-free Mo/ZSM-5 catalyst, 3. FIG. 5 shows that this is also true for the selectivity and yield of BTX after approximately 7 hours on stream.

FIG. 6 shows the XRD patterns for SiC, 6, MCM-22, 7, and Mo/MCM-22/SiC after calcination but before use, 2, demonstrating that the structural integrity of the MCM-22 zeolite and the SiC remains intact after the molybdenum has been loaded, and after the composite material has been calcined.

FIG. 7 shows the XRD patterns for SiC, 6, ZSM-5, 8, ZSM-5/SiC, 9, Mo/ZSM-5/SiC after calcination but before reaction, 4, and Mo/ZSM-5/SiC after use in catalysis, 5. The results demonstrate that the ZSM-5 and the SiC structure remain intact in the composite material after addition of the molybdenum, after calcination, and after use in catalysts.

The plot of FIG. 8 provides the catalytic activity of Mo/ZSM-5/SiC catalysts as a function of the content of different molybdenum-containing species in the catalyst. The plot highlights that catalytic activity, in terms of aromatics yield, appears to be directly correlated with the quantity of isolated Mo species, 10, present in the catalyst. The total Mo content of the catalyst, 11, and the content of nanocrystalline MoO₃ species in the catalyst, 12, appear to be less important factors governing aromatics production.

1. A composition that is catalytically active for methane dehydroaromatisation reactions, which composition comprises a catalyst metal active for methane dehydroaromatisation and a zeolite with Brønsted acidity, which zeolite is selected from those having pores in one or more dimensions with diameters of at least 10 non-oxygen framework atoms, characterised by the composition also comprising silicon carbide.

2. A composition as claimed in claim 1, in which the zeolite is an aluminosilicate zeolite.

3. A composition as claimed in claim 1, in which the zeolite is selected from those having the MWW and the MFI structure.

4. A composition as claimed in claim 3, in which the zeolite is selected from ZSM-5, MCM-22 and MCM-49.

5. A composition as claimed in claim 4, in which the zeolite is ZSM-5.

6. A composition as claimed in claim 1, in which the catalyst metal is selected from one or more of Mo, Re and W.

7. A composition as claimed in claim 6, in which the catalyst metal, or one of the catalyst metals, is Mo.

8. A composition as claimed in claim 1, in which the one or more catalyst metals and optional one or more additional metals are each present in the zeolite at a loading in the range of from 0.1 to 40 wt %.

9. A composition as claimed in claim 1, in which the zeolite content is in the range of from 0.5 to 40% by weight.

10. A process for methane dehydroaromatisation comprising contacting a methane-containing feedstock with a catalyst to produce one or more aromatic compounds and hydrogen, characterised by the catalyst having a composition according to claim 1.

11. A process as claimed in claim 10, in which a methane-containing feedstock is passed over a fixed bed of catalyst at a GHSV of in the range of from 100 to 20 000 mL g⁻¹ h⁻¹.

12. A process as claimed in claim 10, in which the temperature is in the range of from 400 to 900° C., and the pressure is in the range of from 1 to 80 atm.
